(19) United States
(12) Patent Application Publication
(10) Pub. No.: US 2024/0150661 A1
(43) Pub. Date: May 9, 2024

(54) METHOD AND SYSTEM FOR PRODUCING DIESEL FUEL HAVING IMPROVED PROPERTY

(71) Applicants: Naoya YOSHIKAWA, Tokyo (JP); Hiroyasu NAKAMURA, Kanagawa (JP)

(72) Inventors: Naoya YOSHIKAWA, Tokyo (JP); Hiroyasu NAKAMURA, Kanagawa (JP)

(21) Appl. No.: 18/248,304

(22) PCT Filed: Oct. 6, 2021
(86) PCT No.: PCT/JP2021/036923

§ 371 Date: Oct. 13, 2023

(30) Foreign Application Priority Data
Oct. 7, 2020 (JP) 2020-169481

(51) Int. Cl. C10G 32/00; C10G 31/08; C10G 53/02; C12N 9/20; C12N 9/50; C12P 5/00

(52) U.S. Cl. CPC C10G 32/00; C10G 31/08; C10G 53/02; C12N 9/20; C12N 9/63; C12P 5/00; C10G 2300/202; C10G 2300/302; C10G 2300/304; C10G 2300/307; C10G 2300/308; C10G 2400/04

(57) ABSTRACT

[Issues] To provide a method to produce diesel fuel that meets the standards of almost all countries in a short time.

[Solution] A method for producing diesel fuel having improved properties by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, comprises adding a second enzyme solution in light oil having 0.001 to 0.01% by weight of pineapple-derived enzyme containing bromelain, dissolved in light oil to said base oil.

### TECHNICAL FIELD

The invention relates to a method and a system for producing diesel fuel.

### BACKGROUND ARTS

Light oil is generally a petroleum fraction having a boiling point in the range of from about 170 to 370° C., and is used as diesel fuel (hereinafter referred to as Petro diesel), such as fuel for automobile diesel engines, in addition to power generation, agricultural and construction machinery diesel engine fuels, and a fuel for heating such as boilers. In a combustion system where a combustion device, for example, a pyrolysis furnace is used to pyrolyze waste or such to obtain an oil, superheated steam is used as an auxiliary heat source.

Petro diesel is composed of approximately 75% of saturated hydrocarbons (mainly paraffins, including n, iso, and cycloparaffins) and 25% of aromatic hydrocarbons (including naphthalene and alkylbenzenes). Table 1 shows the standards for light oil in Japan,

TABLE 1

| | Kind | | | | |
|---|---|---|---|---|---|
| Item | S1 | 1 | 2 | 3 | S3 |
| Flash point ° C. | 50≥ | 50≥ | 50≥ | 45≥ | 45≥ |
| Distillation Properties of 90% | 360≤ | 360≤ | 350≤ | 330≤*¹ | 330≤ |
| Distillation Temperature (° C.) | | | | | |
| Pour Point (° C.) | +5≤ | −2.5≤ | −7.5≤ | −20≤ | −30≤ |
| Plugging Point(° C.) | | −1≤ | −5≤ | −12≤ | −19≤ |
| Carbon Residue % of 10% | 0.1≤ | | | | |
| Distillation Residue | | | | | |
| Cetane Index *² | 50≥ | 50≥ | 45≥ | 45≥ | 45≥ |
| Kinematic Viscosity(30° C.) | 2.7≥ | 2.7≥ | 2.5≥ | 2.0≥ | 1.7≥ |
| mm²/s(cst) | | | | | |

| | |
|---|---|
| Sulfur Content % | 0.0010≤ |
| Density (15° C.)g/cm³ | 0.86≤ |

When kinematic viscosity (30° C.) is not more than 4.7 mm2/s{4.7 cSt,} distillation properties of distillation temperature is not more than 350° C.

Instead of cetane index, cetane number may be used In addition to Petro diesel, non-petroleum derived alternatives such as biodiesel, BTL, or GTL have been developed. For example, as biodiesel, there are a method for extracting oil by pyrolyzing a carbonaceous raw material previously invented by us, and a method for producing a biodiesel by esterification reaction of oil and fat in the presence of an alcohol utilizing a specific yeast-derived lipase as disclosed in Patent Document 1 (JPA-2002-23339)

In addition to the sulfur content, cetane index, and 90% distillation temperature items, the 2007 revision of the mandatory specification for diesel fuel added six new items as required fuel properties for diesel fuel blended with biodiesel fuel (FAME): fatty acid methyl esters (FAME), triglycerides, methanol, acid number, the sum of gallic acid, acetic acid and propionic acid, and acid number increase. Six additional properties were added for a total of nine (see Table 2).

TABLE 2

| Item | Light Oil Mixed with | | Light Oil Mixed with | |
|---|---|---|---|---|
| | FAME (B5^{mote}) | | No FAME (B0) | |
| Sulfur Content | 0.001 | wt % ≤ | 0.001 | wt % ≤ |
| Cetane Index | 45> | | 45≥ | |
| 90% Distillation | 360° | C.≤ | 360° | C.≤ |
| Temperature | | | | |
| Fatty Acid Methyl Ester | 0.1 | wt % > | 0.1 | wt % < |
| (FAME) | 5.0 [symbol] % ≤ | | | |
| Triglyceride | 0.01 | wt % ≤ | 0.01 | wt % ≤ |

| | | | |
|---|---|---|---|
| Methanol | 0.01 | wt % ≤ | — |
| Acid Value | 0.13 | mgKOH/g≤ | — |
| Sum of Formic Acid, | 0.003 | wt % ≤ | — |
| Acetic Acid and Propionic | | | |
| Acid | | | |
| Increase in Acid Value | 0.12 | mgKOH/g≤ | — |

In Table 2, to distinguish between diesel oil blended with FAME as biodiesel fuel at 0.1 mass % or more and 5 mass % or less, and diesel oil without FAME blending, they are listed as “diesel oil (B5)” and “diesel oil (B0) “Light oil (B5)” and “Light oil (B0)”, respectively.

Similar diesel fuel regulations exist around the world, for example, in Europe, diesel fuel is regulated by EN 590 and biodiesel fuel by EN 14214, as shown in Table 3.

TABLE 3

| Type | | Deisel Fuel | FAME |
|---|---|---|---|
| Corresponding Standard | | EN590: | EN14214: |
| | | 2009/2010 | 2009 |
| Cetane Value | | ≥51 | ≥51 |
| Density (15° C.) | kg/m³ | 820-845 | 860-900 |
| kinematic viscosity (40° C.) | mm²/s | 2-4.5 | 3.5-5 |
| Aromatics | | ≤8.0 | |
| Sulfur Content | mg/kg | ≤10.0 | ≤10.0 |
| Flashing Point | | >55 | ≥101 |
| 10% Carbon Residue | wt % | ≤0.3 | ≤0.3 |
| Ash Content | wt % | ≤0.1 | |
| Water Content | mg/kg | ≤200 | ≤500 |
| Oxidation Stability | g/m³ | ≤25 | |
| FAME Content | volume % | ≤7.0 | ≥96.5 |

Diesel engines using such light oil are advantageous in obtaining good fuel efficiency and realizing high torque, and have the disadvantage that the exhaust gas contains a large amount of NOₓ and PM and, thus, is not clean. Therefore, diesel engines using light oil are subject to various emission regulations.

As an exhaust gas regulation, for example, Euro 5, which will be introduced in the European Union (EU) from 2009, is a stricter exhaust gas regulation for automobiles in consideration of the environment. Euro 5 requests to drastically reduce emission rates, where rate of PM (particulate matter) should reduce 80% (from 0.025 g/km to 0.005 g/km) and NOₓ (nitrogen oxides) should reduce 20% (from 0.25 g/km to 0.2 g/km), in comparison with Euro 4. In the EU, Euro 1, which has already been introduced in 1992, required emission limits for diesel and gasoline vehicles. The standards have subsequently moved to Euro 2 (January 96), Euro 3 (January 2000) and then Euro 4 (January 2005). In particular, Euro 4 is a strict requirement to halve PM and NOₓ in diesel passenger cars and to halve CO (carbon monoxide), hydrocarbons and NOₓ in gasoline passenger cars. Euro 5 will introduce even tougher regulations, which will apply to all new vehicles 18 months after entry into force and all newly registered vehicles 36 months after entry into force. Furthermore, in 2014, a transition to Euro 6, which requires diesel vehicles to meet the same emission standards as gasoline vehicles, is scheduled, and interest in hybrid vehicles, bioethanol fuel, hydrogen fuel, and fuel cells has been increased.

As another measure against exhaust gas from diesel fuel, exhaust gas recirculation is used to take in a portion of the exhaust gas after combustion, mainly for the purpose of reducing nitrogen oxides (NOₓ) in the exhaust gas and improving fuel efficiency at partial load, and re-inhaling it. (Exhaust Gas Recirculation: EGR) system has been proposed.

In addition, as another measure against exhaust gas from diesel fuel, a device for simultaneously removing carbon (CO) and nitrogen oxides (NOₓ) by a three-way catalyst (Three-Way Catalyst, TWC) supporting platinum, palladium, and rhodium.

Furthermore, a selective catalytic reduction (SCR), which converts NOₓ into nitrogen molecules N₂ and water H₂O with a catalyst, has also been proposed as a NOₓ purification device. In particular, an exhaust gas purification system that combines EGR and urea SCR using a urea-based catalyst has been adopted by some major automobile manufacturers, but due to the high cost, other automobile manufacturers are reluctant to adopt it. As a result, it developed into a scandal of camouflaged exhaust gas.

In this way, in addition to retrofitting a diesel engine, Patent Document 2 (Japanese Patent No. 5338268) proposes a low compression ratio clean diesel engine that employs EGR without using a NOₓ catalyst.

As an attempt different from devising the diesel engine side, an attempt to reform the diesel fuel itself has also been made.

In the 1990s, oil supply companies launched premium light oil as gas oil with higher added value than general light oil (normal light oil). Compared to ordinary light oil (normal light oil), to this premium light oil are added a “cleaning agent” that removes dirt from the fuel injection system, and a cetane number improver (cetane number+3) that improves self-ignition (ignition) and reduces white smoke at low temperature start and black smoke at high load. degree), and rust inhibitors (Patent Document 3:JPA-H5-132682).

The cetane number improver is said to be mainly due to the generation of nitro radicals.

In addition, the premium light oil produced by these companies disappeared from the market after 2012 due to reasons such as high cost.

Patent Document 4 discloses a method for producing fuel oil by allowing enzyme water prepared by mixing natural plant complex enzymes with water to be reacted with petroleum oil. According to this method, fuel efficiency can be improved, generation of harmful substances can be easily suppressed, and it is described as being stable.

### PRIOR ART DOCUMENTS

### Patent Documents

Patent Document 1: JPA-2002-233393

Patent Document 2: Japanese Patent No. 5338268

Patent Document 3: JPA-H5-132682

Patent Document 4: Japanese Patent No. 4397432

### SUMMARY OF THE INVENTION

### Problems Solved by the Invention

However, the method according to Patent Document 4 is a method of producing fuel oil from each petroleum oil, and when this is applied to light oil fuel, the reaction time is very long, and the resultant fuel sometimes deviates from standard of Japan, Europe, etc. Also, it is difficult for this method to obtain diesel fuel with stable properties. The performance of the resultant oil varies depending on the properties of the base oil used as the starting material. Furthermore, similar to general diesel fuel, Patent Document 4 cannot apply the problems that suppress exhaust gas such as NOₓ, SOₓ, CO, PM, etc. to make clean exhaust gas, while cleaning exhaust and that improves fuel efficiency by improving combustion efficiency.

Therefore, an object of the present invention is to provide a method for efficiently producing diesel fuel that meets the standards of each country within a short period of time.

Another object of the present invention is to provide a system for efficiently producing diesel fuel that meets the standards of each country within a short period of time.

### Means for Solving Problems

A method of the present invention, which solves the object described above is a method for producing diesel fuel having improved properties by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil.

The present invention also relates to a diesel fuel obtained by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil.

In a specific embodiment of the present invention, there is provided use of pineapple-derived enzyme containing bromelain as a reaction accelerator for adding an oil phase, in the course of the production of diesel fuel by admixing a water phase comprising an aqueous alcohol solution containing lipase enzyme with the oil phase comprising Petoro diesel or crude Petro diesel.

The present invention further relates to a system for producing diesel fuel having improved properties pr comprising: a water phase preparation line having an enzyme mixing tank which mixes lipase enzyme as the first enzyme, a maturation tank having aeration device which matures the mixed solution of water and the lipase enzyme under aeration; and a mixing tank which prepares the prepared aqueous enzyme solution with an alcohol; a base oil preparation line having a base oil tank which supplies a base oil for raw material; and a diesel fuel production line having: a mixing tank which mixes the water phase within the oil phase from the mixing tank which mixes the aqueous phase prepared in the aqueous phase preparation line to maintain a predetermined liquid temperature at a predetermined pressure in the oil phase from the oil phase preparation line; a stirring tank which stirs the oil and water phases mixed in the mixing tank; a pulsing device or vibration device that applies pulse wave or vibration to said oil-water mixture to remove impurities in said oil phase; at least one reaction tank which leaves reacted pulsed or vibrated oil/water mixture standing to cause a reaction; a refinery apparatus which separates the diesel oil from the water phase and refine the diesel oil; and a fuel storage tank which stores the refined diesel oil; the system further adding device for adding the said second enzyme to the base oil in the base oil tank or the mixing tank of the fuel oil production tank.

### Effect of the Invention

According to the present invention, by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil. the resulting diesel fuel conforms to the performance standards of each country, and has improved properties at least one property selected from the group consisting of a cetane number, a cetane index, a polycyclic aromatic content, a flash point, a sulfur content, carbon residue on 10% distillation residue, a pour point, ash weight and viscosity. More specifically, the cetane number (cetane index) and flash point are increased, and the aromatic content, kinematic viscosity, and sulfur content are decreased, resulting in a high-quality diesel fuel.

Even if the base oil which does not meet the standards of each country is used as a starting material, for example, even if a raw material with a cetane index of about 40 or a raw material with a sulfur content of about 0.5% by weight is used, the diesel fuel which meets the standards of each country can be obtained in an amount 1.2 to 1.4 times of base oil.

The reaction time is also 10 to 20% shorter than when the second enzyme is not added to the original oil, and the resulting diesel fue meets the standards of each country.

Furthermore, according to the present invention, there is provided a system for stably producing diesel fuel having improved properties conforming to the standards of each country.

### BRIEF DESCRIPTION OF THE DRAWINGS

FIG. 1 is a flowchart showing the stages of the method for producing diesel fuel of the present invention.

FIG. 2 a flowchart for modifying water from the water source used in the method for producing diesel fuel of the present invention.

FIG. 3 is a schematic view showing one example of the system for producing diesel fuel of the present invention.

FIG. 4(a) and (b) are schematic views each showing embodiments of the diesel fuel production line in the system for producing diesel fuel of the present invention accommodated within a mobile body.

FIG. 5 shows a schematic view showing another example of the system for producing diesel fuel of the present invention accommodated within a mobile body.

### EMBODIMENTS FOR CARRYING OUT THE INVENTION

Now, embodiments of the present invention will be described in detail by referring to the attached drawings.

The terms used in the present invention have the following meanings.

“Petro diesel” is generally a petroleum fraction with a boiling range of approximately 170-370° C., and has properties according the standards shown in Table 1 to Table 3.

“Crude Petro diesel” is incompletely refined Petro diesel, and does not satisfy at least one property among the properties shown in able 1 and Table 2. For example, crude Petro diesel contains sulfur in an amount of about 0.5% by weight.

“Conforming to national standards” means that diesel fuel has properties conforming to the standards of the country to be used. For example, diesel fuel (Petro diesel or diesel fuel composed mainly of Petro diesel) conforming to Japanese standard has properties defined in Table 1 and the diesel fuel conforming to EU standard has properties defined in Table 2.

The diesel fuel obtainable in the production method of the present invention using Petro diesel or crude Petro diesel as a base oil has improved properties in comparison with the base oil and has properties adapted to the standards of countries.

In general, there are many reports on the relationship between diesel fuel properties and exhaust gas characteristics emitted from diesel engines, and cetane number, distillation properties, and aromatic composition are cited as major indicators of fuel properties that have a significant impact. The cetane number is closely related to combustion in the engine and thus affects most exhaust gas components, while the distillation properties are said to affect particulate matter (PM) due to an increase in the heavy content. An increase in aromatic content is said to increase PM emissions and is also believed to further increase NOₓ emissions.

This invention is a method of producing diesel fuel that increases the cetane number, increases the distillation properties, and decreases the aromatic content through multiple enzymatic reactions, thereby reducing particulate matter PM and NOₓ, and increasing the capacity of the original oil by 20% to 40%.

### Production Method

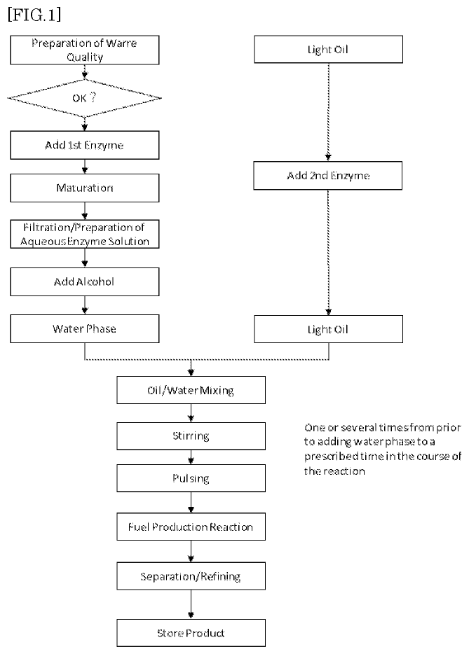

The method of producing diesel fuel of the present invention will now be described by referring to FIG. 1.

As shown in FIG. 1, the method of producing diesel fuel of the present invention is the method for producing diesel fuel for obtaining (upgraded) diesel fuel conforming to the standards of each country. Specifically, first, an aqueous enzyme solution is prepared and a prescribed amount of alcohol is added to the aqueous enzyme solution thus prepared to prepare a water phase. The water phase is mixed and reacted with an oil phase composed of the raw material of Petro diesel to increase an amount of diesel fuel in the oil phase. The oil phase obtained by separation from the water phase through a precision filter to produce diesel fuel for obtaining (upgraded) diesel fuel conforming to the standards of each country.

The first enzyme used in the present invention is a lipase enzyme or a mixture of a lipase enzyme and a cellulase enzyme, and is used for hydrolysis or catalytic decomposition by stirring and mixing with the oil phase described later and subsequent contact. The first enzyme used in the present invention is not restricted as long as it exhibits the function and effect of the present invention and can be selected from the conventional lipase used in the production of biodiesel and the lipase enzyme described in Patent Document 4. Preference is given to use enzyme “SUPER X” commercially available from GGI Worldwide Management Ltd. This enzyme is an enzyme-containing carrier containing about 70-90% by weight of fruit lipase and about 30-10% by weight of cellulase enzyme carried on a zeolite carrier (hereinafter referred to as “first enzyme”).

The water used for preparing this aqueous enzyme solution is preferably soft water having a hardness of 0 to 60 mg/L. It is not preferable to use water having a hardness exceeding 60 mg/L because minerals such as calcium may inhibit enzyme activity.

When the hardness of raw water to be used is so-called hard water exceeding 60 mg/L, it is preferable to perform so-called water softening to make the hardness less than 60 mg/L, preferably less than 30 mg/L. Such water softening is generally performed by a water softening device using a conventionally well-known water filter for water softening.

In addition to water softening, preferred embodiment of the present invention modifies the raw water to be used. Modification applicable to the present invention includes, for example, modification that uses magnetism to break hydrogen bonds in water molecules.

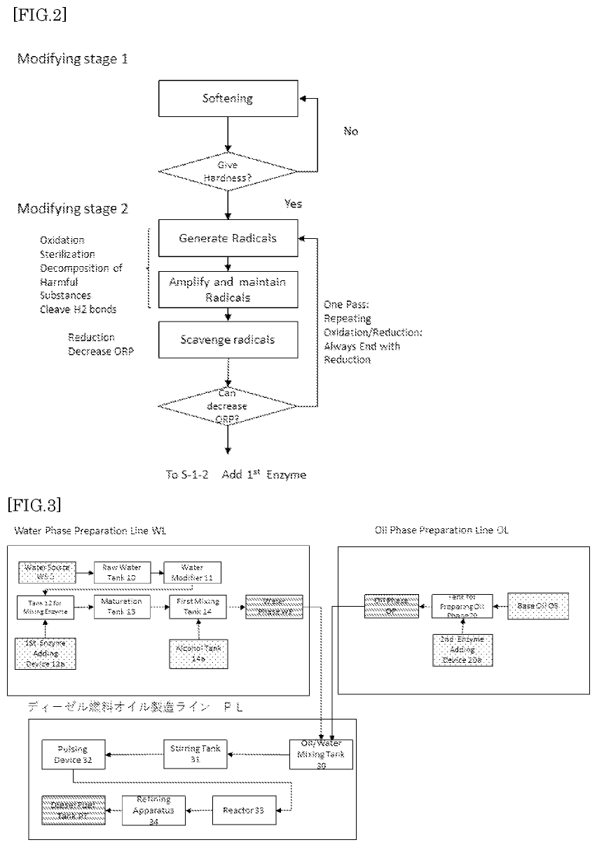

According preferred embodiment of the present invention, as shown in FIG. 2, radicals are applied to water, radicals thus applied are amplified and maintained in the water, and the radials are scavenged. These operations are conducted once or repeatedly.

According to the present inventors, it has been understood that when soft water is passed through an apparatus that repeats the radical generation process, the radical amplification/maintenance process, and the radical scavenge process, microbes in the water are killed, the permeability increases, and the redox potential decreases. It has been found that when the first enzyme is added and matured in such a water environment, the diffusion maturation speed and activation of the first enzyme increase, and the maturation time is shortened by about 10% to 20%. Such an apparatus is disclosed for example in our patent publication JPA2008-15506.

In the present invention, after adding 0.01 to 0.10% by weight of the first enzyme to raw water that has optionally been softened so as to have a hardness of 60 mg/L or less, and modified, the enzymes are activated by maturing for at least 72 hours, with aeration at a temperature of 20-30° C. (room temperature). By maturing under such conditions, the effect of the present invention, that is, enzyme water for the aqueous phase is prepared for reforming and producing diesel fuel that meets the increased amount of base oil and the standards of each country.

In addition, when using an enzyme caried on a zeolite carrier as the first enzyme as in this embodiment, the used zeolite is preferably removed by a filter not so as to be mixed with the oil phase so that the used zeolite does not mix with the oil phase. of the zeolite carrier is removed by a filter, before mixing with the oil phase, preferably at the time of finishing maturation.

In a specific embodiment of the present invention, other enzymes, such as lipase enzymes of different origins and cellulase, can be added prior to or during the maturation process as an auxiliary.

A water phase is prepared by adding and mixing a water-soluble alcohol to the aqueous enzyme solution thus prepared so as to be a 20 to 35% alcohol aqueous solution. Alcohols which can be used herein are not particularly limited as long as they are water-soluble alcohols, and examples thereof include methanol, ethanol (including bioethanol), n-propanol, i-propanol, and mixtures thereof. Methanol is preferred due to its availability and low cost.

On the other hand, the (crude) Petro diesel, which constitutes the oil phase in the present invention and is the base oil, is a petroleum fraction of 170 to 370° C. as described above, and contains about 75% saturated hydrocarbons (mainly n-, iso-, and paraffins, including cycloparaffins), and 25% aromatic hydrocarbons (including naphthalene and alkylbenzene), with impurities such as sulfur, nitrogen compounds, metals, and high-boiling compounds such as asphaltenes.

As described in Patent Document 4, if this is simply reacted with the first enzyme alone, the properties of the diesel fuel obtained will not be constant even if the reaction is performed under the same conditions using the same base oil. In some cases, it became a diesel fuel that did not meet the standards of each country.

This is because Patent Document 4 is mainly aimed at reforming heavy oil and increasing the amount of fuel, and does not consider manufacturing fuel that meets the standards of each country.

When we have made extensive studies so that diesel fuel that meets the standards of each country obtained as a whole while modifying the base oil can be reproduced with good reproducibility, it has been found to be solved by adding a second enzyme dissolved in light oil, and the present invention has been created based on such a technical idea.

This second enzyme is marketed as SUPER X by GGI Worldwide Management Ltd. as a cetane improver for diesel fuel. According to our experiments, when a small amount of this enzyme, which is an agent serving as cetane improver, is added to the oil phase, the startability is improved, especially at low temperatures, the cetane number is increased, the combustion efficiency is increased, and the generation of PM is reduced.

In the present invention, the addition of the second enzyme to the oil phase prior to mixing with the aqueous phase, or at the time of mixing the aqueous and oil phases as described below, or after mixing, in a single or multiple additions totaling 0.0001 to 0.01 mass % to the oil phase, was found to reduce aromatic content by 5 to 10% and increase cetane number by 5 to 10. The cetane number was found to increase by 5-10%. Furthermore, although the amount of NOₓ emissions also depends on the diesel engine used, a 10% improvement was found when applied to marine diesel engines, and a 3-10% improvement was also found for vehicle diesel engines.

In the present invention, the aqueous and oil phases described above are stirred and mixed to bring the first enzyme in the aqueous phase into contact directly or through the interface between the aqueous and oil phases to reform

At this time, the volume ratio of the aqueous phase to the oil phase is 2 to 4:8 to 6, preferably 2.5 to 3.5:7.5 to 6.5, more preferably about 7:3. If the amount of the aqueous phase is more than the following range, it is disadvantageous in terms of water treatment after the reaction and the cost of the alcohol content of the reaction. Conversely, when the amount is less than the above range, the reaction becomes insufficient.

The method of mixing the water phase and the oil phase is to add the water phase into the oil phase at a room temperature (about 25° C.) at a pressure of 2.5 to 3 MPa in a manner that the liquid temperature is kept at a temperature not deactivating the first enzyme (and the second enzyme), i.e., a temperature not exceeding 60° C., preferably not exceeding 45° C. and the mixture is stirred similarly while keeping at a temperature not exceeding 60° C., preferably not exceeding 45° C. The reason why the liquid temperature is kept below 60° C. is that if the temperature exceeds 60° C., the first enzyme may be deactivated.

The method of stirring may be mixing with a blade, but it is preferable to stir by generating a vortex flow from the viewpoint of not generating frictional heat.

After stirring and mixing the oil phase and the water phase in this manner, if desired, the oil-water mixture is subjected to pulse waves by a pulse generator or vibrated by a vibration device to remove residual impurities. Solids and metals are separated and removed via the aqueous phase.

Continuously or intermittently, the oil phase and the aqueous phase are brought into contact with each other by shaking or stirring, and the mixture is allowed to stand at room temperature for several hours, preferably 20 hours or more, more preferably 40 hours or more, to allow reforming and reaction to proceed.

By allowing to stand in this manner, the first enzyme permeates from the water phase to the oil phase from the interface between the oil phase and the water phase, and hydrolyzes and catalytically cracks the hydrocarbons in the raw diesel fuel to produce higher hydrocarbons. As the lower hydrocarbons are formed, lower olefin formation proceeds (reaction from the interface).

On the other hand, due to the action of the second enzyme added to the oil phase, the aromatics in the oil phase are olefinized and successively converted to lower olefins, and elements such as N and S in the aromatic are removed during oil-water separation, via the aqueous phase during oil-water separation.

In this way, both the action of the first enzyme from the oil-water interface and the action of the second enzyme in the oil phase promote lower olefination and produce an upgraded diesel fuel that meets the standards of each country.

The diesel fuel reformed and manufactured in this way has a higher cetane number (cetane index) and flash point, and a lower aromatic content, kinematic viscosity, and sulfur content, resulting in a higher quality diesel fuel.

Even if the base oil is a diesel fuel that does not conform to the standards of each country, for example, even if a raw material with a cetane index of about 40 or a sulfur content of about 0.5% by weight is used, diesel that meets the standards of each country can be obtained in an amount 1.2 to 1.4 times the amount of the base oil.

In the present invention, the interaction between the action of the first enzyme from the interface and the action of the second enzyme in the oil phase reduces the aromatic content by 7.5 to 12% and increases the cetane number by 7 to 12. NOₓ emissions are also expected to improve by the same amount or more as the improvement by the addition of the second enzyme.

### System for Producing Diesel Fuel

Next, the diesel fuel production system (hereinafter referred to as the production system of the present invention), in which diesel fuel is produced by the production method of the present invention, will be described based on FIGS. 3 to 5.

The production system of the present invention mainly comprises an aqueous phase preparation line WL, an oil phase preparation line OL and a diesel fuel production line PL.

The aqueous phase preparation line WL possesses an enzyme mixing tank 12 for adding and mixing raw water from the water source WS and a lipase enzyme as a first enzyme; a maturation tank 13 for preparing an aqueous enzyme solution by maturing the mixed solution of the raw water and the lipase enzyme; and a first mixing tank 14 for preparing an aqueous phase by adding alcohol to the prepared aqueous enzyme solution.

In general, the raw water from the water source WS is stored in the raw water tank 10 and is used after checking whether it meets the raw material standards. Specifically, in the present invention, it is preferable to use soft water having a small amount of minerals such as calcium and magnesium contained in water that inhibit the activity of enzymes.

Generally, soft water is classified as having a hardness of 0 to 100 mg/L, moderately hard water as having a hardness of 101 to 300 mg/L, and hard water as having a hardness of 301 mg/L or more. In the present invention, soft water having a hardness of 60 mg/L or less is preferable.

Therefore, in the present invention, when using water with a high degree of hardness, cations such as calcium ions and magnesium ions contained in the water are reformed by a water softening device (water reforming device 11) having a function of replacing the cations with sodium ions by the action of the ion exchange resin.

When the water from the water source WS, such as tap water, which contains an amount of chlorine and microorganisms more than a predetermined is used as the raw water, it is preferable to modify the water accordingly. It is also within the scope of the present invention to modify the water itself by activating it rather than improving the negative factors contained in the water.

It is preferable to use the aforementioned modification technique shown in FIG. 2) as such an apparatus.

In the system of the present invention, a predetermined amount (0.01 to 0.10% by weight of the first enzyme) of the first enzyme is added to the raw water thus modified as desired in the enzyme mixing tank 12. Then, the aqueous solution to which the enzyme has been added in this manner is matured at room temperature for 72 hours or longer, while being aerated by an aerator (not shown) in the maturation tank 13.

In the system of the present invention, it is required for maturation for 72 hours, so that the maturation tank 13 becomes huge for preparation of a large amount of enzyme solution and production of diesel fuel. For this purpose, this maturation tank 13 can consist of a plurality of tanks 13 arranged in parallel and/or in series.

In the system of the present invention, the aqueous enzyme solution thus prepared in the (final) maturation tank 13 is then transferred to the first mixing tank 14, at which a predetermined amount of alcohol is added. At this time, when the first enzyme used is the enzyme carried on the zeolite carrier as described above, the transfer line to the first mixing tank 14 is equipped with a filter for removing the zeolite carrier. Furthermore, as described above, in the first mixing tank, prior to the addition of alcohol, other enzymes, such as lipase enzymes of different origins, cellulase, etc., may be supplementarily added.

Then, the aqueous enzyme solution to which alcohol has been added is preferably stored in the aqueous phase tank WT while being aerated by an aerator (not shown) until it is used as the aqueous phase.

The oil phase preparation line OL is an oil phase preparation line equipped with a source oil tank to prepare source oil as a raw material. In this embodiment, it is configured with a source oil preparation tank equipped with a second component adding device 20a to add the second enzyme to the source oil that will be the raw material in advance. For example, the second component adding device may be provided in the oil-water mixing tank 30 in the diesel fuel production line PL described below, or in both of them.

In the system of the present invention, the water phase in the water phase tank WT and the oil phase in the oil phase tank OT are separately prepared and stored in the water phase preparation line WL and the oil phase preparation line OL prepared in this manner, and they are mixed and reacted in a diesel fuel production line PL to produce diesel fuel.

The oil-water mixing tank 30 is a tank which first introduces and fills the oil phase in the oil phase tank OT via the pump Po so that the oil phase and the water phase have a predetermined volume ratio, and then pumps the water phase WP to the oil phase thus filled via a pump Pw within a predetermined pressure and temperature range for addition and mixing.

The stirring tank 31 is a tank for stirring the oil/water mixture mixed in the oil/water mixing tank 30. The stirring means at this time can be a blade or the like, but it is preferable to have a stirring device that generates a vortex flow in order to mix the oil and water while controlling heat generation.

The mixed and stirred oil-water mixture is left reacting while keeping the temperature at room temperature for several hours, preferably 24 hours or more, more preferably 48 hours or more, while the oil phase and the water phase are brought into contact with each other by shaking or stirring continuously or intermittently in the reaction tank 34.

Since this reaction also requires a long period of time, it is preferable to adopt a configuration in which the reaction is performed in a plurality of reaction tanks 34, preferably the reaction tanks 34 connected in series.

By allowing to stand in this manner, the first enzyme permeates from the water phase to the oil phase from the interface between the oil phase and the water phase, and hydrolyzes and catalytically cracks the hydrocarbons in the raw diesel fuel to convert higher hydrocarbons into lower hydrocarbons whereby lower olefin formation proceeds (reaction from the interface). On the other hand, due to the action of the second enzyme added to the oil phase, the aromatics in the oil phase are olefinized and gradually converted to lower olefins, while the elements such as N and S in aromatics are removed via the aqueous phase during oil-water separation.

In this way, both the action of the first enzyme from the oil-water interface and the action of the second enzyme in the oil phase promote lower olefination and produce an upgraded diesel fuel that meets the standards of each country. The produced diesel fuel is purified after oil-water separation in a refiner and stored in a diesel fuel tank PT, which is a product tank.

Both the action of the first enzyme from the oil-water interface and the action of the second enzyme in the oil phase promote lower olefination to produce an upgraded diesel fuel that meets national standards. The produced diesel fuel is stored in the diesel fuel tank PT, which is the product tank, after oil and water separated in the refiner.

As described above, using a base oil, the system of the present invention reforms the base oil and can reliably produce diesel fuel that can meet the standards of each country in an amount of about 22.5to 45% by volume relative to the base oil, i.e., 1.2 to 1.4 times % the base oil.

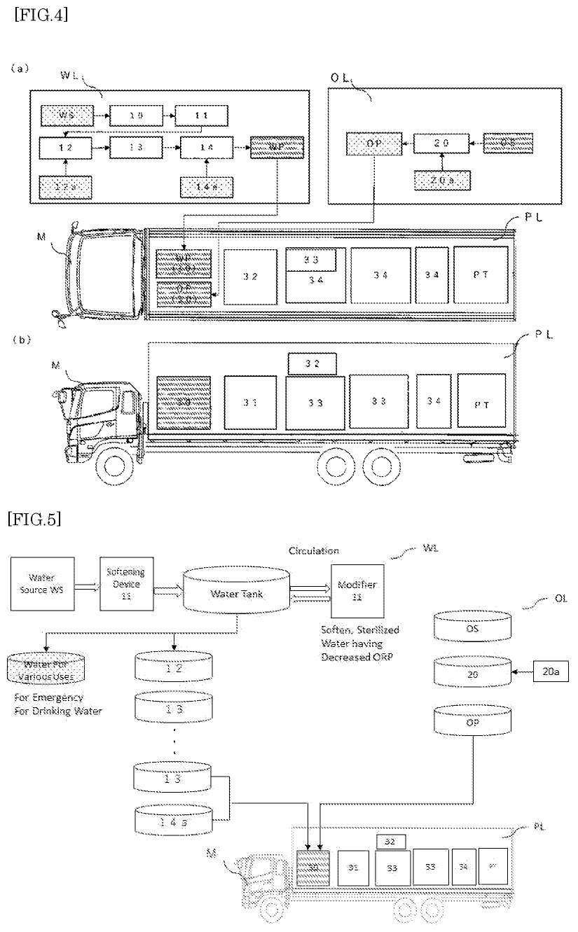

Another embodiment of the system of the present invention will now be described with reference to FIG. 4. Components similar to those in FIG. 3 are denoted by the same reference numerals, and descriptions thereof are omitted.

In the embodiment shown in FIG. 4, it is possible to install the diesel fuel production line PL on the loading platform of the mobile body M, preferably on a transport container.

By configuring in this way, the aqueous phase prepared in the aqueous phase preparation line WL and the oil phase prepared in the oil phase preparation line OL are sequentially put into the mixing tank 30, whereby diesel fuel of the present invention can be produced on-the-go, on-site, or both.

Still another embodiment of the system of the present invention will now be described with reference to FIG. 5. Components similar to those in FIG. 3 and FIG. 4 are denoted by the same reference numerals, and descriptions thereof are omitted.

In this embodiment, a large amount of water is used in the aqueous phase preparation line WL, and therefore the reformed water is used for various purposes, such as water for emergency measures in the event of a disaster, drinking water, domestic water, and agricultural water. etc. can be used in combination.

In this case, it is preferable to apply radicals to water in the reformer 11, amplify and maintain the applied radicals, and then scavenge the radicals once or repeatedly.

It has been found that when soft water is passed through an apparatus that repeats a radical generation process, a radical amplification/maintenance process, and a radical scavenging process, microbes in the water are killed, the permeability increases, and the REDOX potential decreases. When the first enzyme is added and matured in such water environments, the diffusion/maturation speed and activation of the first enzyme are increased, and the maturation time is shortened by about 5% to 10% and the sterilized soft water with low hardness can be used for the water of the aqueous phase preparation line WL of the present invention together with water for various usage.

Although the embodiments of the present invention have been described above, the present invention is not limited to these embodiments. For example, the following items fall within the scope of the present invention:
1 A method for producing diesel fuel having improved properties by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil.

The method according to Item 1 which comprises a maturation stage for maturing the aqueous enzyme solution which adds the first enzyme to soft water having a hardness of 0 to 60 mg/L, and matures the enzyme at a temperature of from 20 to 30° C. with aeration over a period of at least 72 hours, and

a water phase preparation stage for preparing a water phase which comprises mixing the aqueous enzyme solution containing with a water-soluble alcohol.
3 The method according to Item 2, wherein water source is modified prior to use.
4 The method of Item 3, wherein the modification is carried out by softening water so as to have a hardness of 0 to 60 mg/L, by a softening device.
5 The method according to Item 3, wherein said soft water is modified by one or multiple set of treatment of radical treatment and radical scavenge treatment.
6 This method according to any of Items 1 to 5, wherein said first enzyme is an enzyme-containing carrier containing about 70-90% by weight of fruit lipase and about 30-10% by weight of cellulase enzyme carried on a zeolite carrier, and the method comprising removing the zeolite carrier after maturation or preparation of the water phase.
7 This method according to any of Items 1 to 5, wherein the oil phase is mixed with the water phase by adding the water phase to the oil phase with stirring, while maintaining the temperature not exceeding 60° C., to be the volume ratio of the water phase to the oil phase to within the range of 2-4: 8-6;
8 The method according to Item 7, wherein the stirring is conducted in vortex flow.
9 The method according to anyone of Items 1 to 8, wherein after the mixing the oil phase with the water phase, impurities is removed by applying said oil water mixture to a pulse wave or vibration to remove impurities from the oil phase
10 The method according to Item 9, which comprises a reaction stage, which comprises leaving the oil/water mixture at normal room temperature while continuously or discontinuously contacting both phases with vibration or stirring to cause the reaction between the water phase and the oil phase to improve the properties of the oil phase and to produce diesel fuel; and a separation and refining stage, which comprises, after the reaction stage (d), separating the oil phase from the water phase, and refining the separated oil phase by precision filtration.
11 Diesel fuel having improved properties produced by the method according to anyone of Items 1 to 10.
12 The diesel fuel of Item 11, which satisfies at least one of the conditions of Tables 1 to 3.

Also within the scope of the present invention is use of pineapple-derived enzyme containing bromelain as a reaction accelerator for adding an oil phase, in the course of the production of diesel fuel by admixing a water phase comprising an aqueous alcohol solution containing lipase enzyme with the oil phase comprising Petoro diesel or crude Petro diesel.

Furthermore, within the scope of the present invention is a method for modifying diesel fuel by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil.

In addition, the following items are within the scope of the present invention.

A system for producing diesel fuel having improved properties produced by the method according to anyone of Items 2 to 10, comprising: a water phase preparation line having an enzyme mixing tank which mixes lipase enzyme as the first enzyme, a maturation tank having aeration device which matures the mixed solution of water and the lipase enzyme under aeration; and a mixing tank which prepares the prepared aqueous enzyme solution with an alcohol; a base oil preparation line having a base oil tank which supplies a base oil for raw material; and a diesel fuel production line having: a mixing tank which mixes the water phase within the oil phase from the mixing tank which mixes the aqueous phase prepared in the aqueous phase preparation line to maintain a predetermined liquid temperature at a predetermined pressure in the oil phase from the oil phase preparation line; a stirring tank which stirs the oil and water phases mixed in the mixing tank; a pulsing device or vibration device that applies pulse wave or vibration to said oil-water mixture to remove impurities in said oil phase; at least one reaction tank which leaves reacted pulsed or vibrated oil/water mixture standing to cause a reaction; a refinery apparatus which separates the diesel oil from the water phase and refine the diesel oil; and a fuel storage tank which stores the refined diesel oil; the system further adding device for adding the said second enzyme to the base oil in the base oil tank or the mixing tank of the fuel oil production tank.
14 The system for producing diesel fuel according to Item 13, wherein said water phase preparation line possesses a modification apparatus which modifies the raw water. for softening the raw water to soft water having a hardness of from 0 to 60 mg/L.
15 The system for producing diesel fuel according to Item 15, wherein said water phase preparation line possesses a modification apparatus which modifies the raw water.
16 The system for producing diesel fuel according to claim 15, wherein said modification apparatus applies oxidation and reduction to decrease a reduction oxidation potential and to cleave the hydrogen bonds of water.
Claim 17 The system for producing diesel fuel according to any one of Items 14 to 16, wherein the raw water modified in the aqueous phase preparation line is shared with other uses.
18 The system for producing diesel fuel according to any one of Items 13 to 17, wherein said system for producing diesel fuel is composed of a stationary, installed, aqueous phase preparation line, a stationary or movably mounted, oil phase preparation

### DESCRIPTION OF SYMBOLS

WL Water phase preparation line
WS Water source
WP Water phase
OL Oil phase preparation line
OS Base oil
OP Oil phase
PL Diesel fuel production line
PT Diesel fuel tank
M Mobile body
Material tank
11 Water modification device
12 Enzyme mixing tank
12a Device for adding first enzyme
13 Maturation tank (Tank for preparing aqueous enzyme solution)
14 First mixing tank (Tank for mixing water and alcohol)
14a Alcohol tank
20 Base oil preparation tank (second mixing tank)
20a Device for adding second enzyme
30 Oil/water mixing tank
31 Stirring Tank
32 Pulsing device
33 Reactor
34 Purification device

1. A method for producing diesel fuel having improved properties by mixing an aqueous enzyme solution containing 0.01 to 0.1% by weight of a lipase enzyme as a first enzyme with a water-soluble alcohol so as to be an aqueous alcohol solution having an alcohol concentration of from 20 to 35%, which comprises adding a second enzyme solution in light oil having 0.001 to 0.01% by weight of pineapple-derived enzyme containing bromelain, dissolved in light oil to said base oil.

2. The method according to claim 1 which comprises a maturation stage for maturing the aqueous enzyme solution which adds the first enzyme to soft water having a hardness of 0 to 60 mg/L, and matures the enzyme at a temperature of from 20 to 30° C. with aeration over a period of at least 72 hours, and
a water phase preparation stage for preparing a water phase which comprises mixing the aqueous enzyme solution containing with a water-soluble alcohol.

3. The method according to claim 2, wherein water source is modified prior to use.

4. The method of claim 3, wherein the modification is carried out by softening water so as to have a hardness of 0 to 60 mg/L, by a softening device.

5. The method according to claim 3, wherein said soft water is modified by one or multiple set of treatment of radical treatment and radical scavenge treatment.

6. This method according to any of claims 1 to 5, wherein said first enzyme is an enzyme-containing carrier containing about 70-90% by weight of fruit lipase and about 30-10% by weight of cellulase enzyme carried on a zeolite carrier, and the method comprising removing the zeolite carrier after maturation or preparation of the water phase.

7. This method according to any of claims 1 to 5, wherein the oil phase is mixed with the water phase by adding the water phase to the oil phase with stirring, while maintaining the temperature not exceeding 60° C., to be the volume ratio of the water phase to the oil phase to within the range of 2-4: 8-6;

8. The method according to claim 7, wherein the stirring is conducted in vortex flow.

9. The method according to anyone of claims 1 to 8, wherein after the mixing the oil phase with the water phase, impurities is removed by applying said oil water mixture to a pulse wave or vibration to remove impurities from the oil phase

10. The method according to claim 9, which comprises a reaction stage, which comprises leaving the oil/water mixture at normal room temperature while continuously or discontinuously contacting both phases with vibration or stirring to cause the reaction between the water phase and the oil phase to improve the properties of the oil phase and to produce diesel fuel; and a separation and refining stage, which comprises, after the reaction stage (d), separating the oil phase from the water phase, and refining the separated oil phase by precision filtration.

11. Diesel fuel having improved properties produced by the method according to anyone of claims 2 to 10.

12. The diesel fuel of claim 11, which satisfies at least one of the following properties:
TABLE 4

| | Kind | | | | |
|---|---|---|---|---|---|
| Item | S1 | 1 | 2 | 3 | S3 |
| Flash point ° C. | 50≥ | 50≥ | 50≥ | 45≥ | 45≥ |
| Distillation Properties of 90% | 360≤ | 360≤ | 350≤ | 330≤*¹ | 330≤ |
| Distillation Temperature (° C.) | | | | | |
| Pour Point (° C.) | +5≤ | −2.5≤ | −7.5≤ | −20≤ | −30≤ |
| Plugging Point(° C.) | | −1≤ | −5≤ | −12≤ | −19≤ |
| Carbon Residue % of 10% | 0.1≤ | | | | |
| Distillation Residue | | | | | |
| Cetane Index*² | 50≥ | 50≥ | 45≥ | 45≥ | 45≥ |
| Kinematic Viscosity(30° C.) | 2.7≥ | 2.7≥ | 2.5≥ | 2.0≥ | 1.7≥ |
| mm/s(cst) | | | | | |
| | |
|---|---|
| Sulfur Content % | 0.0010≤ |
| Density (15° C.)g/cm³ | 0.86≤ |

13. A system for producing diesel fuel having improved properties produced by the method according to anyone of claims 2 to 10, comprising:
a water phase preparation line having an enzyme mixing tank which mixes lipase enzyme as the first enzyme, a maturation tank having aeration device which matures the mixed solution of water and the lipase enzyme under aeration; and a mixing tank which prepares the prepared aqueous enzyme solution with an alcohol;
a base oil preparation line having a base oil tank which supplies a base oil for raw material; and
a diesel fuel production line having:
a mixing tank which mixes the water phase within the oil phase from the mixing tank which mixes the aqueous phase prepared in the aqueous phase preparation line to maintain a predetermined liquid temperature at a predetermined pressure in the oil phase from the oil phase preparation line;
a stirring tank which stirs the oil and water phases mixed in the mixing tank;
a pulsing device or vibration device that applies pulse wave or vibration to said oil-water mixture to remove impurities in said oil phase;
at least one reaction tank which leaves reacted pulsed or vibrated oil/water mixture standing to cause a reaction;
a refinery apparatus which separates the diesel oil from the water phase and refine the diesel oil; and
a fuel storage tank which stores the refined diesel oil;
the system further adding device for adding the said second enzyme to the base oil in the base oil tank or the mixing tank of the fuel oil production tank.

14. The system for producing diesel fuel according to claim 13, wherein said water phase preparation line possesses a modification apparatus which modifies the raw water. for softening the raw water to soft water having a hardness of from 0 to 60 mg/L.

15. The system for producing diesel fuel according to claim 15, wherein said water phase preparation line possesses a modification apparatus which modifies the raw water.

16. The system for producing diesel fuel according to claim 15, wherein said modification apparatus applies oxidation and reduction to decrease a reduction oxidation potential and to cleave the hydrogen bonds of water.

17. The system for producing diesel fuel according to any one of claims 14 to 16, wherein the raw water modified in the aqueous phase preparation line is shared with other uses.

18. The system for producing diesel fuel according to any one of claims 13 to 17, wherein said system for producing diesel fuel is composed of a stationary, installed, aqueous phase preparation line, a stationary or movably mounted, oil phase preparation.
